(19) United States
(12) Patent Application Publication
(10) Pub. No.: US 2005/0279369 A1
(43) Pub. Date: Dec. 22, 2005

(54) Method and apparatus for the treatment of presbyopia and glaucoma by ciliary body ablation

(76) Inventor: J. T. Lin, Oviedo, FL (US)

Correspondence Address: J. T. Lin, 4532 Old Carriage Trail, Oviedo, FL, 32765, US

(21) Appl. No.: 10/871,903

(22) Filed: Jun. 21, 2004

(51) Int. Cl. A61F 9/013

(52) U.S. Cl. 128/898; 623/905

(57) ABSTRACT

Laser and non-laser means to remove a portion of the ciliary body tissue for the treatment of presbyopia and glaucoma are disclosed. Mechanisms based on elasticity increase the sclera-ciliary-body and zonule “complex” is proposed. Total accommodation based a lens relaxation and lanes anterior shift is calculated. The preferred embodiments for the ablation patterns include radial lines, curved lines, ring dots or any non-specific shapes in a symmetric geometry. The surgery apparatus includes lasers in UV (0.19 to 0.35 micron) and IR (2.8 to 3.2) micron, and non-laser device of radio frequency wave, electrode device, bipolar device and plasma-assisted device. Post-operation medication such as pilocarpine (0.5%-5%) or medicines with similar to reduce postoperative regression or enhance the accommodation is presented. A much deeper, about (0.8-1.4) mm, ablation depth supraciliary body is proposed for (50%/-200%) greater accommodation than the prior arts based on superficial scleral ablation or expansion.

### BACKGROUND OF THE INVENTION

1. Field of the Invention

The present invention relates to method and apparatus for the treatment of presbyopia and glaucoma by changing the rigidity property of the sclera-ciliary-zonus complex to lower the intraocular pressure or increase the accommodation of treated eye.

2. Prior Art

Corneal reshaping including a procedure called photorefractive keratectomy (PRK) and a new procedure called laser assisted in situ keratomileusis, or laser intrastroma keratomileusis (LASIK) have been performed by lasers in the ultraviolet (UV) wavelength of (193-213) nm. The commercial UV refractive lasers include ArF excimer laser (at 193 nm) and other non-excimer, solid-state lasers such as those proposed by the present inventor in 1992 (U.S. Pat. No. 5,144,630) and in 1996 (U.S. Pat. No. 5,520,679). The above-described prior arts using lasers to reshape the corneal surface curvature, however, are limited to the corrections of myopia, hyperopia and astigmatism.

Refractive surgery using a scanning device and lasers in the mid-infrared (mid-IR) wavelength was first proposed by the present inventor in U.S. Pat. Nos. 5,144,630 and 5,520,679 and later proposed by Telfair et al. in U.S. Pat. No. 5,782,822, where the generation of mid-IR wavelength of (2.5-3.2) microns were disclosed by various methods including: the Er:YAG laser (at 2.94 microns), the Raman-shifted solid-state lasers (at 2.7-3.2 microns) and the optical parametric oscillation (OPO) lasers (at 2.7-3.2 microns).

Corneal reshaping may also be performed by thermal coagulation conducted by a Ho:YAG or diode laser (at about 2 microns in wavelength) proposed by Sand in U.S. Pat. No. 5,484,432, or by conductive keratoplasty (CK) using a radio frequency thermal energy. These methods, however, were limited to low-diopter hyperopic corrections. Strictly speaking, these prior arts did not correction the true “presbyopia” and only performed the mono-vision for hyperopic patients. A thermal beam (or energy) is required and the treated area was inside the limbus and within the optical zone diameters of about (8-10) mm.

The above prior arts, however, did not actually resolve the intrinsic problems of presbyopic patient caused by age where the cornea lens loss its accommodation as a result of loss of elasticity due to age.

All the above-described prior arts are using methods to change the cornea surface curvature either by tissue ablation (such as in UV laser) or by thermal shrinkage (such as in Ho:YAG laser) and all are using thermal of non-thermal energy onto the central potion of the cornea.

The direct method for presbyopia correction, therefore, is to increase the accommodation of the presbyopic patients by changing the intrinsic properties of the sclera and ciliary tissue to increase the lens accommodation without changing the cornea curvature. Because there is no reshaping of the cornea, the treated eye shall keep its original far vision while its near vision is improved under a presbyopia treatment. This is the fundamental difference between corneal reshaping and sclera-ciliary tissue ablation.

To treat presbyopic patients using the concept of expanding the sclera by sclera expansion band (SEB) has been proposed by Schachar in U.S. Pat. Nos. 5,489,299, 5,722,952, 5,465,737 and 5,354,331. These mechanical approaches have the drawbacks of complexity and are time consuming, costly and have potential side effects. To treat presbyopia, the Schachar U.S. Pat. Nos. 5,529,076 and 5,722,952 propose the use of heat or radiation on the corneal epithelium to arrest the growth of the crystalline lens and also propose the use of lasers to ablate portions of the thickness of the sclera. However, these prior arts do not present any details or practical methods or laser parameters for the presbyopic corrections. No clinical studies have been practiced to show the effectiveness of the proposed concepts. The concepts proposed in the Schachar U.S. Pat. Nos. 5,354,331 and 5,489,299, regarding lasers suitable for ablating the sclera tissues were incorrect because he did not identify which lasers are “cold lasers”. Many of his proposed lasers are thermal lasers that will cause thermal burning of the cornea, rather than tissue ablation. Furthermore, the clinical issues, such as locations, patterns and depth of the sclera tissue removal were not indicated in these prior patents. In addition, it is essential to use a scanning or fiber-coupled laser to achieve the desired ablation pattern and to control the ablation depth on the sclera tissue. Schachar's methods proposed in his prior arts also require the weakening of the sclera and increasing of lens diameter for patients accommodation. The new mechanisms proposed by the present inventor in U.S. Pat. No. 6,263,879 (Lin-879), on the contrary, propose that lens diameter decreases and anteriorly shifted when accommodation occurs to see near. In addition, no implant is needed in the Lin-879 invention (based on non-expansion theory), which is required in Schachar's based on expansion theory.

Another prior art proposed by Spencer Thornton (Chapter 4, “Surgery for hyperopia and presbyopia”, edited by Neal Sher (Williams & Wilkins, MD, 1997) is to use a diamond knife to incise radial cuts around the limbus areas. It requires a deep (90%-98%) cut of the sclera tissue in order to obtain accommodation of the lens. This method, however, involves a lot of bleeding and is difficult to control the depth of the cut that requires surgeons' extensive skill. Another drawback for presbyopia correction provided by the above-described incision-method is the major postoperative regression of about (30%-80%). We note that there is intrinsic difference between the ablation-method proposed In this invention and the knife-incision-method. The sclera space produced by the incision-method is not permanent (unless implantation like Schachar is used) and this space will be reduced during the tissue healing and cause the regression. This is the major source of regression in incision-method and in Schchar's SEB method.

The prior arts of the present inventor, U.S. Pat. Nos. 6,258,082 and 6,263,879 and PCT/US01/24618 (the “Lin-082-879”) proposed the use of a laser to remove portion of the sclera tissue based on the concept of “lens relaxation”, where the scleral ablation causes the ciliary body to contract for lens relaxation to see near. From our clinical results using the method proposed in our prior arts, we found that there are two major drawbacks: first, regression is improved (less than that of incision method and SEB), but still significantly reduce the efficacy for postoperation after 9 to 12 months; secondly, the initial accommodation amplitude (M) ranging from 0.5 to 2.5 diopter (with a mean about 1.9 diopter) is too low when postoperative regression of (20%-40%) is included. In addition, our clinical data also showed the total failure in some cases, which is the accommodation amplitude (AA) after surgery is less than 0.5 diopter with Jaeger (J) reading higher than 5. The acceptable J-reading is J=(1.0 to 3.0) for near vision at about 40 cm. A successful treatment for typical patients shall reduce the preoperative J-reading (about 5 to 7) such that a Snellen near value of 20/32 (or J3) or better is achieved. For severe presbyopia with preoperative J=(10 to 15), a successful treatment shall expect J=(3 to 5), postoperatively. If minor regression of (5% to 15%) is allowed, a successful treatment will require an initial AA of about (1.8 to 3.5) diopters. The prior arts of Lin-082-879 failed to meet the above criteria for those cases with regressions or those cases with lower initial AA (say, less than 1.2 diopter) after laser sclera ablation.

One objective of the present invention, therefore, is to provide an apparatus and method to obviate the drawbacks in the prior arts, in particular, to improve the initial efficacy or initial M value and reduce the postoperative regression.

It is yet another objective of the present invention to provide new mechanisms that support minimum regression and improved efficacy by ciliary body ablation, rather than scleral ablation proposed by Lin's prior arts.

It is yet another objective of the present invention to provide a theoretical modeling and calculation for accommodation amplitude (AA) and the principles behind the technology presented in the invention.

It is yet another objective of the present invention to provide parameters for ablation patterns and depth required for sufficient accommodation.

It is yet another objective of the present invention is that outflow of the vitreous is improved to reduce the abnormally high intraocular pressure (IOP) of glaucoma patients.

A further objective is to provide a treatment for presbyopia.

A further objective is to provide a treatment for primary open angle glaucoma.

Further objectives of the invention will become apparent from the description of the invention to be detailed as follows.

### SUMMARY OF THE INVENTION

A two-component theory consisting of lens relaxation and lens anterior shift is proposed. For maximal accommodation, ciliary body (CB) ablation is proposed and is analyzed by an elastic model defined by contraction momentum and a replacement. The preferred tissue ablation means include laser and non-laser energy. The preferred ablation total depth is about (0.8 to 1.4) mm outside the limbus.

It is yet another preferred embodiment is that CB is ablated without ablating the conjunctiva layer or sclera layer.

It is yet another preferred embodiment is that the ablation patterns on the sclera area include radial lines, curved lines, ring-dots or any non-specific shapes in a symmetric geometry.

It is yet another preferred embodiment is to use post-operation medication such as pilocarpine or medicines with similar nature that may cause ciliary body contraction to stable and/or enhance the post-operative results after the ablation-method.

It is yet another preferred embodiment is to use post-operation medication such as pilocarpine or medicines with similar nature that may cause ciliary body contraction to stable and/or enhance the post-operative results after the ablation-method. Further preferred embodiments of the present invention will become apparent from the description of the invention that follows.

### BRIEF DESCRIPTION OF THE DRAWINGS

FIG. 1 shows the ocular structure of a human eye (a side view).

FIG. 2 shows the effect of ciliary body contraction causing lens radius change and anterior shift.

FIG. 3 shows the comparison of scleral and ciliary body ablation.

FIG. 4 shows these preferred embodiments for ciliary body ablation.

FIG. 5 shows a modeling for accommodation mechanisms and its efficiency.

FIG. 6 shows various ablation patterns outside the limbus.

### DETAILED DESCRIPTION OF THE INVENTION AND THE PREFERRED EMBODIMENTS

A surgical system in accordance with the present invention comprises a tissue removal or ablation means includes electromagnetic wave such as a coherent wave (or laser), and non-laser wave such as radio frequency (RF) wave, electrode device, bipolar device and plasma assisted electro-surgical device. When a RF devices are used, the preferred embodiment requires a minimum thermal damage to the sclera and ciliary tissue with efficient ablation which can be controlled by its frequency (10 to 1000) KHz, pulse duration, 100 microseconds to continuous wave, and its power output (0.1-20) W. The “ablation” is defined in a general sense to include tissue removal by means of excision or evaporation. The dimension of the removed tissue, its depth, width and length are controlled by the energy, peak power, fluency and spot size of the energy wave. When a laser is used, we also require efficient tissue ablation with minimal thermal damage. Therefore, the preferred laser wavelength is the region where tissue (or water) has strong absorption, or small penetration depth. These include laser spectrum in UV (0.19 to 0.35) micron and in infrared (IR) range of (2.8 to 3.2) micron. Other ranges of spectrum will not have effective may also ablate tissues effectively via its mega-watt peak power. Therefore, our preferred laser shall also include the above described short pulsed laser. For lasers in our selected UV or IR range, the preferred pulsed duration is between one nanosecond and 300 microsecond. Lasers with continuous wave (CW) mode at low peak power with too much thermal damage to the tissue should be avoided.

We shall first describe our theory behind the invention for the increase accommodation amplitude (AA) to treat presbyopia. It has been known that presbyopia was caused by age. However, the complete description for the mechanisms of accommodation is not conclusive, which includes capsular theory (von Helmholtz theory), lens-crowding theory (Schachar theory), Catenary theory (by Coleman, Ophthalmology, 2001, vol. 108, page 1544-51) and the two-component elastic theory (by the present inventor). All those theories, however, have a common principle that ciliary theory (CB) contraction causes lens accommodation for near vision, although lens power increase (or curvature change) may attribute to various mechanisms: via measure gradient between vitreous and aqueous compartments (Coleman), via relaxation of the lens capsule (Helmoholtz), or via combination of lens relaxation and anterior shift (Lin). Therefore, the fundamental issue for presbyopia treatment becomes how to improve or enhance the CB contraction which causes the increase of system power or lens power. The prior arts of SEB (Schachar) and laser scleral ablation (Lin) both are dealing with the superficial layer of the sclera tissue, by sclera expansion or by Increasing the elasticity of the laser ablated scleral regions. The change of scleral structure is then translated to the movement (or contraction) of the ciliary body (CB) and the zonular fiber connected to the lens. Based on prior arts of Schachar and Lin, the influence from sclera superficial change to CB, then to zonus and lens is rather inefficient due to the ocular structure of the CB-zonule-lens complex and the “remote” distance from the sclera layer to the zonule and lens. The present invention proposes to ablate “directly” the CB tissue which is closer to the zonus-lens and therefore will be much more efficient. It was reported in a “stretching experiment” (Glasser and Campbell, Vision Research, vol. 38, p. 209-229) that each one mm contraction of CB may induce (0.8 to 2.6) diopter of accommodation in young lens (age 10-53), but almost no power change for old lens (age 54 to 87). This clinical data also supports our theory that lens anterior chamber shift (ACS) shall play an important role, particularly in old eyes.

To calculate the total accommodation amplitude (AA), we propose the “Lin dynamic model” by introducing two components, the anterior shift (AS) and lens relaxation (LR) to 87). This clinical data also supports our theory that lens anterior chamber shift (ACS) shall play an important role, particularly in old eyes.

To calculate the total accommodation amplitude (M), we propose the “Lin dynamic model” by introducing two components, the anterior shift (AS) and lens relaxation (LR) AA=AS+LR=M1(dS)+M2(dR), where lens power change is converted to AA by a factor, CF=(0.7-0.8), rather than 100% conversion. Our calculations (Lin, to be published in Journal of Cataract and Refractive Surgery, 2005) showed that one mm dS of the lens will cause about M1=(1.0 to 1.7) diopter of image myopic shift (for patients to see near) and the reversed process, posterior shift (PS) will allow the patient to see far. We note that these AS and PS are “dynamical” effects allowing the lens to move forward and backward for a presbyopic patient to accommodate both near and distance vision. The second component LR causes the presbyopic lens to see near by lens relaxation with decreased radii of the lens, mainly by the anterior capsule of the lens. For a typical post-surgery patients with an average accommodation amplitude (M) of +2.0 D, we propose that the AA may attribute to AS or LR or the combination of them, depending on lens age (or its rigidity).

Our numerical calculation showed an increase of AA=2.0 D may be achieved by any of the following: (a) lens relaxation (dR) with decreased radius R1 from 10.56 to 9.0 mm without AS; (b) combining dR with R1 decreased to 9.5 mm (with a lens power change of 1.36 D and system power increase of 1.02 D) and dS of 0.65 mm (with a system power increase of 0.98 D); and (c) an anterior shift (AS) of about 1.33 mm without dR, assuming m1=1.5(D/mm). Although the lens power change is very sensitive to its radii changes, about (0.8 to 2.7) D/mm as shown by our calculations, its effect on AA however is limited by the rigidity of the lens nucleus or capsule (RLC) and the available amount of ciliary contraction and its spacing to the lens, and the zonular effective length (ZEL) defined by the amount of expansion to allow lens central curvatures (radii) change. The AA given by anterior lens shift (AS) on the other hand is not limited or affected by the condition of RLC, therefore it is still possible to have sufficient AA (say +1.0 D to 1.5 D) by a pure AS without LR, particularly for lens with initial radii smaller than 9.5 mm. noting that AA is inverse proportional to the initial value of (R1, R2).

Clinically, it is import to note that the total accommodation amplitude (AA) is governed by the amount of ciliary body contraction, therefore the M shall be governed by the location and the amount (or volume) of ciliary body (CB) tissue removed, rather than just the depth or length of the ablation. A minimum threshold (TH) of ciliary body tissue must be removed in order to have efficient M for the patient to see near at about 40 cm. From the empirical data of Glasser A and Campbell MCW (Presbyopia and the optical changes in the human crystalline lens with age. Vis Res Vol 38:209-229, 1998) and our calculated data, we find that the change of accommodation amplitude (M) versus ciliary body contraction distance (C) is non-linear in nature. The AA per mm change of C, or Mc, depends on the value of C as follows: Mc=(1.94, 2.3, 2.6, 1.0, 0.8) D/mm, for C ranges of C=(0.0-0.5), (0.6-1.0), (1.1-1.5), (1.5-2.0) and (2.0-2.5) mm. Based on these data, we estimate that C=(0.4 to 0.7) mm will be needed for a typical M=(1.8 to 3.5) diopter which is defined as a successful treatment. The prior arts based on superficial sclera ablation (Lin-879) fail to achieve this criteria, particularly after regression. The CB ablation in the present invention which is fundamentally different in the location, structure and ablation depth, therefore will be (50% to 200%) more efficient than prior arts. Furthermore, “pseudo-accommodation” (with major regression) was reported in prior arts of SBE due to globe axial elongation after the treatment, which may also occur in Lin-879. True accommodation most likely to occur in the present CB ablation method, where the ablation location has the right structure associate to the zonule and lens. The superficial scleral expansion (SEB) or ablation (Lin-879) causes the increase of sclera ring radius, but it is very inefficient in affecting the CB contraction. These major arts also suffer major regression due to sclera healing, as clinically reported.

We note that without the above theoretical calculations and modeling, it would be very difficult to predict the accommodation amplitude and the new mechanisms based on LR and AS. Our method in this invention and parameters for the proposed device and clinical techniques are based upon the above theoretical findings. Further analysis on the mechanisms and efficiency of ciliary body contraction will be discussed based on FIG. 1 to FIG. 5 as follows.

FIG. 1 shows the diagram of a human eye (a side view). The ocular structure of an eye 11 consists of the cornea 12, the iris 13, the lens 14, the limbus 15, the conjunctiva 16, the sclera 17, the choroids layer 18, the ciliary body (CB) 19 which is connected to the lens 14 by the zonule 20. The lens shape and its location (or the anterior chamber depth) are governed by the tensile force from sclera-ciliary-zonule and the pressure (or pressure gradient) in the anterior chamber 21 and in the vitreous 22. The typical values of these ocular components are about: (0.5-0.7) mm for total thickness of conjunctiva and sclera layer; (0.6 to 1.4) mm for the thickness of CB having length about (4.5 to 5.5) mm; the limbus is located at about (5.5 to 6.0) mm from the center of the cornea. From this diagram, we propose the following mechanisms for efficient accommodation, or large value of accommodation amplitude (AA) which is given by our 2-component theory M=AS (anterior shift)+LR (lens relaxation). Both AS and LR are proportional to the amount of CB contraction (CT) which is limited by the movement elasticity of the sclera-CB-zonule complex (SCZ) and the spacing between each of these components. Therefore “loosening” of sclera, CB or zonule will enhance CT which is significantly reduced for aged eyes.

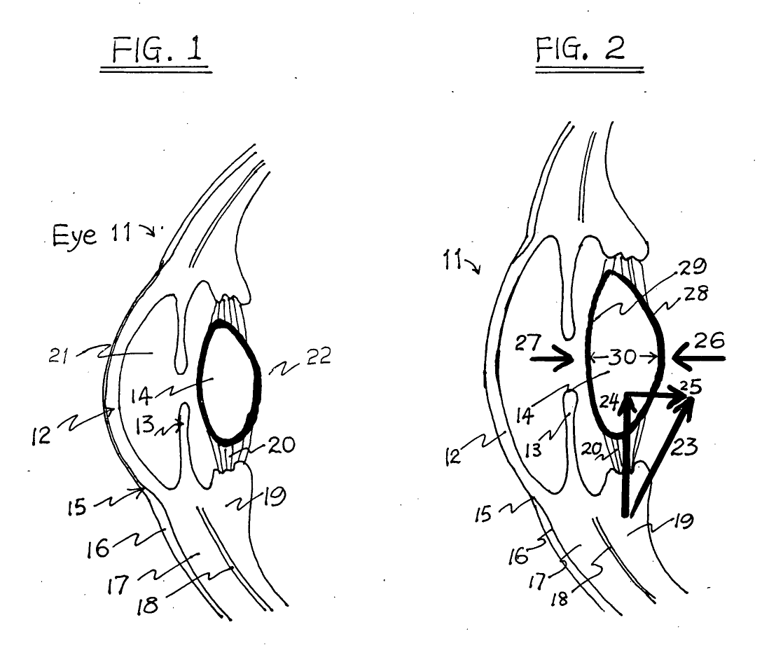

The relationship among CT, AS and LR during accommodation is further illustrated in FIG. 2. The CB contraction force vector 23 has two components, along the zonule lens director 24 and perpendicular to the lens axial 25, where the horizontal force 25 stimulates an increasing (or reaction) pressure pulse 26 toward the lens posterior surface 28 (LPS), whereas a drop of pressure pulse 27 induced against the anterior lens surface 29 (ALS). Therefore CB contraction causes the lens forward displacement (or AS) and the decreasing of the radius of curvature (or increasing lens power) of LPS and ALS, the lens thickness 30 is also increased at the accommodative state. Based on the above described mechanisms, we are able to further analyze the efficiency of CB contraction (or AA). We defined the value of CT by a momentum defined by P=MV=M(d/t), where M is the mass of the SCZ complex which moves at a speed of V (to move a distance d) during the accommodation or CB contraction period of t. It was reported by Coleman et al. (Ophthalmology 2001, vol. 108, p. 1544-51) that the response time was about t=0.5 second in an electrical stimulation of CB of a primate eye for a rise of the vitreous pressure pulse. Now we may use the concept in the present invention that CB contraction speed (V) or its distance (d) is inverse proportional to the mass of the SCZ complex (M) for a given momentum (P) or contraction force to analyze the AA or CB contraction efficiency as follows.

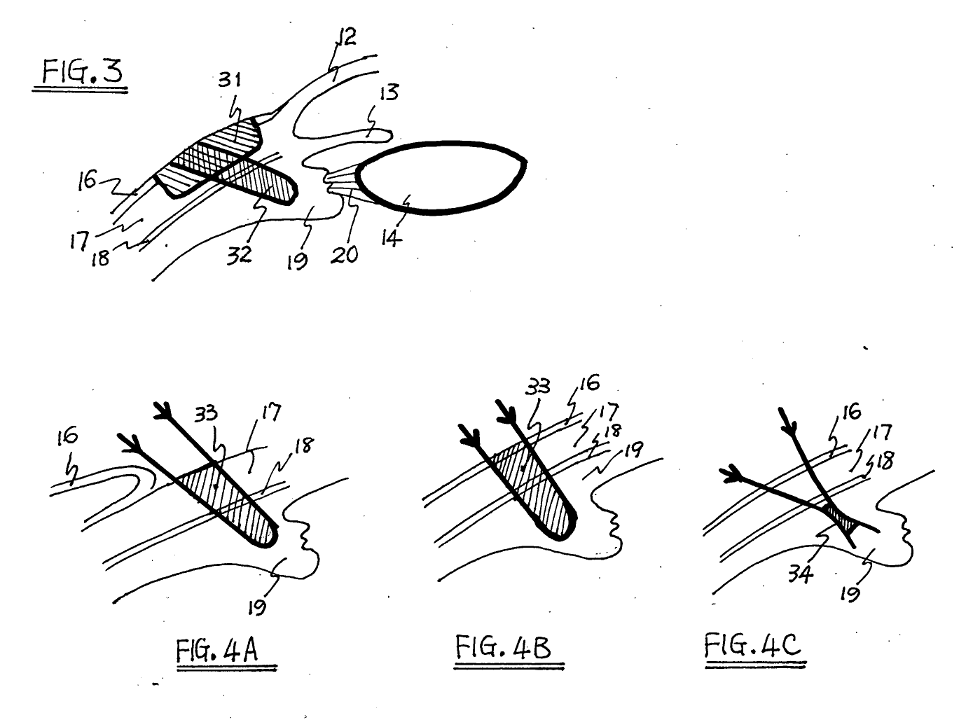

As shown in FIG. 3, the sclera ablation area 31 is superficial with a depth about 80% of the sclera thickness or about (0.4-0.5) mm, as proposed by the prior patents of Lin (U.S. Pat. Nos. 6,258,082, 6,263,879) and PCT/US01/24618. These prior arts also require an ablation length about 4.5 mm and a width about 0.5 mm (the spot size of the laser). In contrast to the prior arts, the present invention has the following parameters: the ablated area 32 is much deeper, about (1.0 to 1.3) mm, ablation length is shorter, about (0.5 to 1.5) mm, and width about (0.5 to 1.0) mm, or spot size of the ablative laser. Lin's prior patents required sclera ablation depth shallower than its thickness to avoid the choroids layer to be ablated and to avoid perforation in the area where CB is thin. Because of the ablation length of about 4.5 mm out from the limbus, most of the ablation area proposed in Lin's prior patents has thin CB, about (0.3 to 0.5) mm, and perforation of choroids and CB may occur if ablation depth is not carefully controlled. In the present invention, we propose the ablation area is closer to the limbus, within (0.5 to 1.5) mm where the CB is thicker, about (1.0 to 1.3) mm, to avoid perforation, whereas allows a deeper CB ablation for higher efficiency.

FIG. 4 illustrates examples of the preferred embodiments in this invention. As shown in 4-A, the CB sclera tissue is ablated after preparing a flap of the conjunctiva layer 16(CL), that is the CL 16 is intact for fast tissue refilling of the ablated area 33. FIG. 4-B shows another preferred embodiment where the CL 16 is also ablated together with the sclera layer 17 and CB 19 with the advantage of being faster and simple procedure without the time consuming process in preparing the CL flap. The third example shows the advantage of having both the CL 16 and sclera 17 intact and only CB 19 is ablated, where a laser beam needed to be tightly focused inside the central portion of the CB in order to generate the ablation area 37. Another requirement is that the laser shall be highly transparent to water or CL, sclera and CB tissue when it is not tightly focused. In other words, the laser shall have a spectrum about (0.8 to 1.3) micron and operated at a short pulsed mode (less than 10 nanosecond) and can be focused to a spot about (0.1 to 0.5) mm in diameter to generate high peak power. The preferred commercial lasers for example 4C include short pulse Nd:YLF or Nd:YAG laser. The preferred laser for processes shown in Examples of 4-A and 4-B shall include UV lasers of ArF (at 193 nm), XeCl (at about 308 nm) and solid-state laser Nd:YAG, Nd:YLF with frequency up-converted by nonlinear crystals such as BBO, LBO, KDP, KTP to wavelength of (193 to 335)nm; and infrared lasers with wavelength of (2.8 to 3.2) micron include Er:YAG (at 2.94 micron), Er.YSGG (at about 2.8 micron) and optical-parametric-oscillation (OPO) down-converted solid-state lasers of Nd:YAG or Nd:YLF at about (2.8 to 3.2) micron. The pulse width of these UV and IR lasers preferred to be between 1.0 nanosecond and 300 microsecond with a repetition rate of at least 5 Hz and laser spot size (at the conjunctiva or sclera surface) about (0.2 to 1.0) mm. A preferred total ablation depth of about (0.8 to 1.4) mm, preferably about (1.0 to 1.2) mm, that is the CB layer is about 50% in depth ablated with a 50% margin (or about 0.6 mm) intact to avoid perforation, noting that the normal CB thickness (at its maximal) is about (1.0 to 1.4) mm, mean of 1.2 mm, whereas patients with glaucoma has thinner CB thickness of about (0.6 to 0.9) mm per reported data of Okamoto et al. (Am J Ophthalmology, 2004, vol. 137, p. 858-862). The preferred-total ablation depth is about (0.4 to 0.8) mm below the choroids layer.

Another important clinical aspect of the present invention is that (50% to 200%) greater accommodation efficiency (AE) may be achieved by the CB ablation than the superficial sclera ablation, to be shown below. Given the same momentum of CB contraction, P1=P2 for CB and sclera ablation with the mass of SCZ complex and ciliary-zonule complex M1 and M2, and displacement d1 and d2, respectively, we obtain M1(d1)M2(d2), so d1=(M2/M1)(d2). Because M2 is about (50% to 200%) larger than d2, that is the CB ablation may achieve an AE 50% to 200% greater than that of sciera ablation. We note that the mass of SCZ complex after sclera ablation (M2) is much larger than that of CB ablation (M1) where M1 and M2 are defined as the mass of the portion which can move in response to CB contraction after ablation. In CB ablation, M1 is much smaller than M2 due to the fact that the “loosened” portion is closer to zonule. Furthermore, the ablated “loosened” tissue inside the CB is much closer to the zonule than the remote superficial sclera ablation. This geometry factor also enhances the AE in CB ablation method (CBA). Regress in sclera-ablation-method due to tissue healing and the large inertial mass (M2) of SCZ is also greatly reduced in CBA. In both cases, more flexible fibrous tissue (from sub-conjunctiva) may grow and fill-in the ablated areas, and causes the SCZ to be contracted easier than tissue prior to ablation. Greater detail based on a mechanical model is shown as follows.

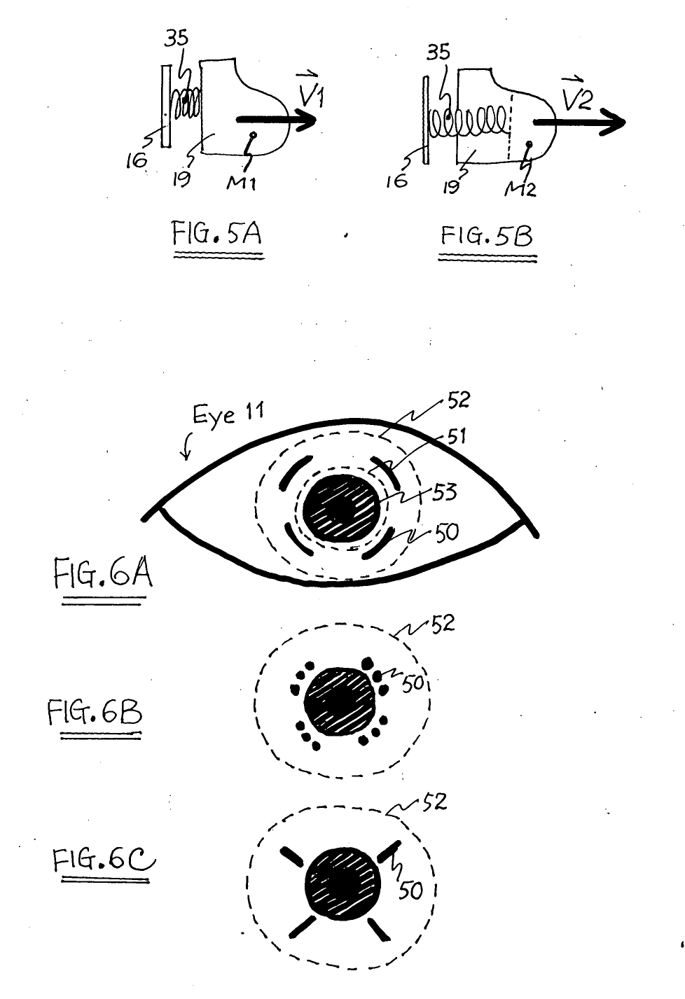

To demonstrate the accommodation efficiency, we use a modeling shown in FIG. 5. For the sclera ablation, the “loosened” scleral area is modeled as the spring 35. Connecting the conjunctiva 16 to the ciliary body 19 having a mass of M1 and a contracting speed of V1 (FIG. 5A). As a comparison, FIG. 5B shows parameters for ciliary body ablation method (CBA). The longer spring 35 stands for a deeper ablation of the CB 19 having a much smaller mass M2 and higher t2=(M2/M1)V1 as discussed earlier. Other than the “mass effect”, the ocular structure also demonstrate that CB ablation which is closer to the lens shall be much more efficient than that of scleral superficial ablation.

The preferred ablation patterns shall include radiation lines, curved-lines, spots and any unspecified patterns within the region defined by a radial distance of from about 6 mm to about 9 mm from the center of the cornea (or optical axis), or most preferably within a region of (6.5 to 7.0) mm when pattern of curved lines is used. FIG. 6 shows the examples of the preferred patterns, where the ablation region 50 is outside the limbus 53 and in the area between two circles 51 and 52 having a diameter of about 12 mm and 18 mm, respectively. It will be understood that the forgoing illustrations simply provide few examples of the ablation patterns, any other unspecified patterns and numbers of spots, lines, shall be also included, as far as they are symmetric to the optical axis. To avoid perforation, one preferred embodiment is to have ablation along the maximal thickness direction of CB, which is about (30 to 50) degree, most prefer off the normal incident of the conjunctiva surface and about (1.0 to 1.5) mm away from the limbus as shown in FIG. 1.

It has been clinically shown that sclera expansion (by SEB) or laser sclera ablation may reduce the intraocular pressure (IOP) particular for subject with elevated IOP. Therefore the CB ablation method in this invention shall achieve the same. The IOP reduction may be resulted from the increase of the pore size in the trabecular meshwork after tissue ablation of expansion.

In Lin's prior patent of laser sclera ablation, the ablation depth is controlled to be less than the sclera thickness (about 0.6 mm) to avoid penetration of the choroids. The CB ablation in this invention is to ablate deep into the CB layer (which is about 1.2 mm thick). To keep about (0.4 to 0.6) mm intact depth of the CB, one preferred embodiment is to control the laser spot size about (0.2 to 1.0) mm, most preferably of (0.5 to 0.7) mm, with energy per pulse about (3 to 10) mJ (for UV laser) and (5 to 20) mJ (for IR laser), such that the ablation depth per pulse is about (10 to 30) microns. For a typical laser repetition rate of (5 to 20) Hz, a total ablation depth of 1.2 mm (including 0.6 mm of conjunctiva-sclera layer and about 50% of CB layer) will take about (3 to 5) seconds, which can be easily controlled by a footswitch with a (0.2 to 0.5) second off and on response time. Depending on the types of laser used and its spot size on eye surface, we may calibrate the most preferably energy per pulse and its repetition rate, such that the CB ablation procedure can be performed within reasonable time (say 5 to 15 minutes per eye), whereas the ablation rate is not too fast to be controlled by a footswitch to avoid perforation and keep ablation depth precision within about 0.1 mm. For example, lower laser repetition rate of 5 Hz will ablate the tissue 4 times slower than that of 20 Hz and is easier to control the depth without perforation of CB. Another preferred embodiment is to use high repetition rate (15 to 20) Hz at the initial ablation and adjust to lower rate, (5 to 10) Hz when ablation is about 70% completed, or after the choroids layer is ablated. Ablation depth control is more critical in CB ablation than that of sclera ablation, which is superficial and perforation is less concerned.

The preferred embodiments for laser energy delivered to the eye surface shall include: (a) computer controlled scanning means such as motorized galvometer, (b) articulated arm; (c) optical fiber. For UV lasers, delivery means (a) and (b) are preferred because it is hard to find highly transparent fiber materials, whereas optical fiber is preferred for IR lasers. The preferred articulated arm or fiber shall also include a hand piece with its end piece can be removed and sterilized for multiple uses. In addition, the ablation patterns and depth proposed in this invention may be produced by software controlled, motorized or manually.

Another preferred embodiment is not to open the conjunctiva layer, but to insert the fiber tip through the conjunctiva layer and ablate the sclera and CB tissue underneath such that the procedure is done non-invasively. To do this procedure, the conjunctiva layer may be lifted to generate the “gap” for fiber tip to insert into the gap and ablate the desired patterns underneath. Additional advantages of this non-invasive method is to avoid or minimize bleeding or infection. We note that most of the bleeding is due to cutting of the conjunctiva tissue rather than the ablation of the sclera tissue. As discussed in FIG. 4, we have also presented 3 preferred examples for the ablation of CB.

The preferred embodiment for the non-laser methods shall include, but not limited to, physical blades or knife, electromagnetic wave such as radio frequency wave, electrode device, bipolar device and plasma assisted electrode device. The electromagnetic wave generator is commercially available. However, the parameters of the device such as its frequency, pulse duration and repetition rate and the size of the electrode tip shall be selected for efficient cutting (or ablation) with minimum thermal damage to the tissue to be removed.

Another preferred embodiment is to use post-operation medication such as pilocarpine (0.5%-5%), most preferable 1%, or medicines with similar nature which may cause ciliary body contraction. The post-operation medicine will cause more stable, less regression and/or enhancement after the treatment. The total initial accommodation short after the procedure using the medicine shall include the tissue removal effects and the effect due to medicine (contraction). Long terms results shall be mainly due to tissue removal. The initial ciliary body contraction trigger or enhancement is important for stable long terms results and to prevent regression caused by tissue healing

While the invention has been shown and described with reference to the preferred embodiments thereof, it will be understood by those skilled in the art that the foregoing and other changes and variations in form and detail may be made therein without departing from the spirit, scope and teaching of the invention. Accordingly, threshold and apparatus, the ophthalmic applications herein disclosed are to be considered merely as illustrative and the invention is to be limited only as set forth in the claims.

1. A method of removing a portion of the ciliary body tissue of an eye comprising the steps of:
selecting a tissue removal means; and
controlling said tissue removal means to remove said ciliary body in a predetermined pattern and in a predetermined area of an eye outside the limbus, whereby patient's accommodation amplitude increases to improve near vision.

2. A method of claim 1, wherein said accommodation amplitude increase is caused by the change of the elastic property of the sclera-ciliary-zonule complex.

3. A method of claim 1, wherein said accommodation amplitude increase is caused by the contraction of said complex.

4. A method of claim 1, wherein said accommodation amplitude increase is caused by lens relaxation or changing of lens curvatures.

5. A method of claim 1, wherein said accommodation amplitude increase is caused by lens anterior shift.

6. A method of claim 1, wherein said accommodation amplitude is further enhanced or stabilized by post-operation medicine including (0.5% to 5%) pilocarpine or medicines in similar nature.

7. A method of claim 1, wherein said tissue removal means includes a laser having a wavelength of about (0.19-0.35) micron or about (2.5-3.2) micron, a pulse duration of about 1.0 nanosecond to about 300 microsecond and a spot size of about (0.2-1.0) mm on the eye surface.

8. A method of claim 7, wherein said laser is an excimer laser of ArF or XeCl.

9. A method of claim 7, wherein said laser is an Nd:YAG, or a Nd:YLF, or an Er:YAG, or an Er:YSGG, or an OPO laser.

10. A method of claim 7, wherein the energy of said laser is delivered to said predetermined area by an optical fiber, or a scanning device, or an articulated arm.

11. A method of claim 1, wherein said tissue removal means includes an electromagnetic wave at radio frequency ranging of (10-1000) KHz and power of (0.1-20) W.

12. A method of claim 1, wherein said tissue removal means includes an electrode device, or a bipolar device, or plasma assisted electrode device.

13. A method of claim 1, wherein said predetermined pattern includes radial lines, or curved lines, or spots or any non-specific shapes.

14. A method of claim 1, wherein said predetermined area includes area outside the limbus defined by the area between two circles having diameters of about 12 mm and 18 mm.

15. A method of claim 1, wherein said predetermined pattern has a depth about (0.8-1.4) mm and is controlled manually or by computer software.

16. A method of claim 1, wherein an intraocular pressure of the eye is reduced.

17. A method of claim 1, wherein said ciliary body is ablated about 50% of its thickness together with the ablation of conjunctiva and sclera layer of an eye.

18. A method of claim 1, wherein said ciliary body is ablated without ablating the conjunctiva layer.

19. A method of claim 1, wherein said ciliary body is ablated without ablating the conjunctiva layer or the sclera layer.
